(19) United States
(12) Patent Application Publication
(10) Pub. No.: US 2006/0193800 A1
(43) Pub. Date: Aug. 31, 2006

(54) Variable tint cosmetics

(76) Inventors: John Reinhardt, Riverside, CA (US); John Hill, Mesa, AZ (US); Michele Ward, Queen Creek, AZ (US); J. Steve Brown, Gilbert, AZ (US)

Correspondence Address: NOBLITT & GILMORE, LLC., 4800 NORTH SCOTTSDALE ROAD, SUITE 6000, SCOTTSDALE, AZ, 85251, US

(21) Appl. No.: 11/340,297

(22) Filed: Jan. 26, 2006

Related U.S. Application Data
(60) Provisional application No. 60/647,531, filed on Jan. 26, 2005.
(60) Provisional application No. 60/647,532, filed on Jan. 26, 2005.

(51) Int. Cl. A61K 8/02; A61K 8/97

(52) U.S. Cl. 424/63; 424/74

(57) ABSTRACT

The disclosed systems and methods for delivery of variable tint pigment materials in cosmetic concealer/foundation products generally include anhydrous carrier particles adapted to carry pigment. The pigmentation carrier particles may be optionally admixed with anhydrous (optionally tinted) base formulation material that is suitably adapted to suspend and/or separate the pigmentation carrier particles. Disclosed features and specifications may be variously controlled, adapted or otherwise modified to realize improved site-specific delivery of pigment in customized concentrations in order to achieve a desired result. Exemplary embodiments of the present invention generally provide anhydrous pigmentation carrier particles that may be mixed with substantially anhydrous topical base formulations that allow a user to deliver a selected concentration of concealer/foundation tint to a specific region of the skin, for example, in order to obscure a blemish.

### RELATED APPLICATIONS

This application claims the benefit of U.S. Provisional Patent Application Ser. No. 60/647,531 filed in the United States Patent and Trademark Office on Jan. 26, 2005 by John Reinhardt, John Hill, Michele Ward, and J. Steve Brown, and U.S. Provisional Patent Application Ser. No. 60/647,532 filed in the United States Patent and Trademark Office on Jan. 26, 2005 by John Reinhardt, John Hill, Michele Ward, and J. Steve Brown.

### FIELD OF INVENTION

The present invention generally relates to cosmetic preparations; and more particularly, representative and exemplary embodiments of the present invention generally concern cosmetic formulations suitably adapted to deliver variable amounts of pigment to different application areas of the skin.

### BACKGROUND OF INVENTION

Traditional tint makeup systems, such as liquid foundations and concealers, obscure skin imperfections by covering the skin surface with a layer of pigments. These pigments typically contain a substantially homogenous and even dispersion of the desired pigmentation material throughout the product. The color of the pigments generally depends on the type of skin imperfection to be covered. Conventional liquid foundations contain a blend of pigments which are selected to ideally match the natural skin tone of the user. This generally requires that a consumer choose the most suitable tint from a large number of possible tints, each designed to match a specific skin tone.

Typically, liquid foundations contain from about 8% to 16% total pigment. Of this material, roughly 80% is titanium dioxide (whitener), 15% yellow iron oxide, 5% red iron oxide and 1% black iron oxide. This general formula may be suitably adjusted to reproduce any given number of natural skin tones.

While conventional liquid foundations enable the user to achieve a relatively even tone over the majority of the coverage area, they do not generally allow the user to shade a particular area more lightly or more darkly at will. The user may lay down a thicker or thinner coat in certain areas, but the tint will generally remain substantially the same.

Conventional concealer products are available in either liquid or powder form and may be used in tandem with conventional foundations to neutralize the color of skin imperfections having a significantly different color than that of the user's otherwise unblemished skin tone. These conventional concealers generally operate to counter the color of a blemish by covering it with a layer of pigment, which is typically oppositely colored with respect to the color of the blemish. For example, a red blemish may be neutralized by a green pigment, resulting in an overall tan tone.

In the field of cosmetic, personal care and pharmaceutical products, emollient compositions are usually employed as agents to soften or smooth the skin by reducing roughness, cracking and/or irritation. In general, smoothing is believed to be effected by the penetration of the emollient into the surface layers of skin tissue, through the action of rubbing and massaging upon application by the user. The ancient Greek physician, Galen, is thought to have made one of the first emollients consisting of beeswax, spermaceti, almond oil, borax and rosewater.

Emollients tend to be bland, fatty, oleaginous substances which may be applied locally to the skin, mucous membranes or abraded tissue. One of the benefits of emollients lies in their ability to exclude water-soluble irritants, as well as air and air-borne bacteria when a layer of emollient is present. At present, there are numerous ingredients which may be employed to function as emollients in a variety of products, with a variety of component ingredients that may act in subtly different ways. For example, some emollients rest on the surface of the skin and generally serve to impede water loss from the skin. These materials are generally comprised of large organic molecules that form a hydrophobic barrier to generally prevent water from leaving the surface of the skin. Examples of such emollients are lanolin, mineral oil, silicone derivatives and petroleum jelly.

There are several emollient substances that are commonly employed in cleansing and antiphlogistic creams and lotions. Compound ointment bases, creams and other medical applications are also materials that find use as emollients. Among the more common conventional emollient materials are castor oil, corn oil, cottonseed oil, rose water ointment, apricot kernel oil, avocado oil, grape seed oil, hazelnut oil, olive oil, sesame oil, theobroma oil, almond oil, myristyl alcohol, and recently other natural oils such as jojoba oil.

Other ingredients that have been used as emollients include a number of fatty acids derived from either plant or animal sources. Fatty acids generally comprise aliphatic hydrocarbon or other organic chains with carboxylic substituents thereon, typically having between 8 and 24 carbon atoms in the chain backbone. Fatty acids have been used in creams, lotions, shaving creams, lipsticks and as pressing agents in pressed powders and blushes. Fatty acids, which are typically used in cosmetics formulations, generally include at least one of stearic acid, oleic acid, myristic acid and palmitic acid. Other typical fatty acids include linoleic acid, behenic acid and other common fatty acids of the general formula CₙH₍₂ₙ₊₁₎COOH.

Fatty alcohols have been found to be less sticky and less heavy than many other fatty materials (such as fatty acids), and are frequently used to improve the viscosity and stability of lotions and creams. Fatty alcohols also have utility in reactive hair dying and perming products. Examples of fatty alcohols which find use in the field of cosmetics and personal care products are cetyl alcohol, lauryl alcohol, stearyl alcohol and oleyl alcohol.

Additional examples of emollients are fatty esters. One of the qualities of fatty esters is that they generally do not feel as oily to the touch as some other types of fatty emollient ingredients. Examples include isopropyl palmitate, isopropyl myristate and glyceryl stearate.

Another emollient is jojoba oil, which is generally derived from the seed of the species Simmondsia chinensis. Jojoba is a seed oil with excellent skin feel. The oil is composed almost exclusively of wax esters, with little or no triglycerides present. A major portion of the commercial production of jojoba oil is used by the cosmetic industry as an emollient in a variety of products.

### SUMMARY OF THE INVENTION

In representative aspects, the present invention provides compositions and methods for delivering customized amounts of pigment to an application area of the skin using a variable tint cosmetic concealer and/or foundation formulation.

Jojoba ester compositions have been found to function well as a dry (e.g., anhydrous) carriers or vehicles for the application of materials to the skin of consumers. These esters have been found to be useful in pure or blended forms as carriers in the personal care, cosmetic and pharmaceutical fields of use. Jojoba esters may be provided with a range of properties (from the composition of the ester itself or from additives and blended materials) and may be suitably adapted to provide improved feel when used in conjunction with other conventional carriers, vehicles, bases, actives and additives. Upon application and rubbing of the compositions, the jojoba ester based compositions leave the skin feeling soft (which is typical of high quality emollients), yet provide a mildly persistent coating which carries the actives without leaving a wet or oily feel to the skin of the user.

Advantages of the present invention will be set forth in the Detailed Description which follows and may be apparent from the Detailed Description or may be learned by practice of exemplary embodiments of the invention. Still other advantages of the invention may be realized by means of any of the instrumentalities, methods or combinations particularly pointed out in the claims.

### DETAILED DESCRIPTION OF EXEMPLARY EMBODIMENTS

The following representative descriptions of the present invention generally relate to exemplary embodiments and the inventors' conception of the best mode, and are not intended to limit the applicability or configuration of the invention in any way. Rather, the following description is intended to provide convenient illustrations for implementing various embodiments of the invention. As will become apparent, changes may be made in the function and/or arrangement of any of the elements described in the disclosed exemplary embodiments without departing from the spirit and scope of the invention.

Various representative implementations of the present invention may be applied to any system for providing a variable tint concealer and/or foundation product for use in cosmetic applications. As used herein, the terms “bead”, “particle”, “sphere”, or any contextual variant thereof, are generally intended to include anything that may be regarded as at least being susceptible to characterization as, or generally referring to a discrete formulation component taken either alone or in combination with a carrier solution.

A detailed description of an exemplary application, namely a composition and method for applying a variable tint concealer and/or foundation product to an area of the skin, is provided as a specific enabling disclosure that may be generalized to any application of the disclosed composition and method for customized cosmetic pigment delivery or color application to the skin, in accordance with various representative embodiments of the present invention.

The present invention describes an effective anhydrous delivery composition for use in personal care and cosmetic products. The composition is essentially solid at room temperature, may be provided in various shapes and sizes (especially as spheres or beads), and may be produced from combinations of fatty alcohols, isopropyl esters and wax esters obtained from the oil contained in the seed of the jojoba plant (Simmondsia chinensis).

These emollient compositions generally preserve the excellent skin feel attributed to jojoba oil, as well as increasing the range of applications for cosmetic formulations that may be suitably adapted to utilize emollients that are generally more polar and hydrophilic than those found in naturally occurring jojoba oils. Exemplary compositions, in accordance with various representative embodiments of the present invention, generally form stable emulsions much more readily than those employing naturally occuring jojoba oil. Exemplary compositions may further provide excellent emolliency to otherwise normally dry cosmetic pigmentation systems, with the emollient acting as a pigment wetting agent as well as an aid to application of a smooth and even distribution of the dry system. Representative compositions in accordance with the instant invention may also be adapted to function as excipients in, for example, pressed powders.

Exemplary compositions comprising fatty alcohols, isopropyl esters and jojoba wax esters (jojoba oil) may be obtained by the base catalyzed alcoholysis reaction between jojoba oil and an alkyl alcohol. In the alcoholysis reaction, examples of the base catalyst materials include, but are not necessarily limited to, metal alkoxides, alkali metal alkoxides, inorganic hydroxides, alkali metal hydroxides, and the like; such as, for example, NaOCH₃ (sodium methoxide), NaOCH₂CH₃ (sodium ethoxide), as well as potassium, calcium and lithium counterparts, KOH, and NaOH (e.g., anhydrous alkali metal hydroxides in alcohol solution, with the alcohol of the solution generally employed as the alcohol used in the reaction). See, for example, U.S. Pat. No. 6,280,746 for a detailed description of representative chemical processes that may be employed to obtain suitably adapted jojoba ester carrier compositions.

In an exemplary embodiment, the present invention enables the user to not only adjust the overall tint of the foundation/concealer system during application, but also to control where to darken or lighten the tint over the application area (typically the face). In another exemplary embodiment, in accordance with a representative aspect of the present invention, the disclosed composition and method generally enables a user to more precisely conceal specific areas of the skin with improved accuracy, without substantially disrupting the tone of the surrounding areas. Thus, the disclosed system may be suitably adapted to serve several functions, such as, for example, that of: a concealer, a foundation, a shadowing agent, a liquid make-up, a powder make-up, and/or the like.

In general, an exemplary variable tint cosmetic product may include at least two components: an optionally tinted base formulation (similar to traditional tinted moisturizing creams); and soft carrier particles (e.g., beads or microspheres) containing a suitable concentration of pigment material(s). The carrier particles may be suspended substantially evenly in the base formulation, or may be provided substantially separate from the base formulation. The carrier particles may be suitably adapted to deliver pigment onto the skin as they are rubbed, or otherwise mechanically manipulated in the desired area, to abrade and release the pigment onto the skin. This in turn allows the user to achieve a customized hue, saturation and/or brightness of desired color.

The pigmentation carrier particles may be soft, spherical and large enough to be noticed both visually as well as tactilely. The particles may remain in suspension with the base formulation or in isolation prior to use. The present invention anticipates that pigmentation carrier particles may have an average diameter of up to about 100 microns to more than about 4000 microns.

In an exemplary and representative application, the pigmentation carrier particles may carry up to about more than 10% total pigment, and may be suitably adapted to deliver the pigment to the skin as the carrier particle is mechanically abraded by, for example, rubbing or massaging action applied by the user. In another exemplary application, the system may provide a formulation for a variable tint foundation product, with the pigmentation carrier particles loaded with, for example, up to about more than 10% total iron oxide pigment. In a representative embodiment, in accordance with an exemplary aspect of the present invention, the pigmentation carrier particles may be suitably adapted to leave substantially no debris (e.g., shell structure) behind after use.

In a representative and exemplary application, the base formulation may comprise conventional (optionally tinted) moisturizer base with a small amount of skin tone pigment dispersed substantially homogenously throughout. An exemplary variable tint cosmetic product may representatively comprise the base formulation with the pigmentation carrier particles in substantially stable suspension. In another representative and exemplary application, the base formulation may be a foundation base, generally comprising conventional liquid foundation base with titanium dioxide. A smaller amount of darker pigments (e.g., enough to match the lightest skin tones) may also be present.

The user may manipulate or move the pigmentation carrier particles suspended in the base formulation (or otherwise provided separately in isolation) in order to neutralize the color of, for example, a blemish and/or to conceal skin imperfections. As soon as the desired concealment is achieved, the user may pick or brush off the remaining pigment particles from the surface of the skin and dispose of them, for example, with a tissue or cofton swab, etc. In a representative and exemplary application, the pigment carrier particle and base formulation may preferably have an extended “dry down” (e.g., “play”) time during application (as compared with conventional concealer products), in order to allow the user to fully manipulate the pigmentation carrier particles to achieve a desired effect.

An alternative exemplary embodiment of the disclosed variable tint cosmetic system may employ a base formulation that is used substantially separately and/or alongside unsuspended (i.e., neat) pigmentation carrier particles. One or more pigment colors may be used, such as, for example: green to cover red blemishes; yellow/white to cover dark rings under the eyes; purple to cover yellow marks; and/or the like. Neat pigmentation carrier particles may be applied substantially directly onto the skin by the user, rubbed onto the surface of the blemish, and then covered with, for example, a conventional foundation product. This may be particularly useful in the case where the user has more than one type of blemish requiring more than one type of concealer, or if the user wishes to better control the accuracy and intensity of the concealing pigment's application.

Although the specifically enabling embodiments disclosed herein suggest the use of jojoba ester beads as the delivery vehicle for carrying the pigment material(s), any anhydrous particle formulation may be alternatively, conjunctively or sequentially employed, including, for example: ethylhexyl palmitate, petrolatum (VASELINE), polyethylene, and/or the like.

In the foregoing specification, the invention has been described with reference to specific exemplary embodiments; however, it will be appreciated that various modifications and changes may be made without departing from the scope of the present invention as set forth herein. The specification is to be regarded in an illustrative manner, rather than a restrictive one and all such modifications are intended to be included within the scope of the present invention. Accordingly, the scope of the invention should be determined by the claims and their legal equivalents rather than by merely the examples described above.

For example, the steps recited in any method or process embodiment may be executed in any order and are not limited to the specific order presented in the claims. Additionally, the components and/or elements recited in any apparatus or composition embodiment may be assembled or otherwise operationally configured in a variety of permutations to produce substantially the same result as the present invention and are accordingly not limited to the specific configuration recited in the claims.

Benefits, other advantages and solutions to problems have been described above with regard to particular embodiments; however, any benefit, advantage, solution to problem or any element that may cause any particular benefit, advantage or solution to occur or to become more pronounced are not to be construed as critical, required or essential features or components of the invention.

As used herein, the terms “comprising”, “having”, “including” or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present invention, in addition to those not specifically recited, may be varied or otherwise particularly adapted to specific environments, manufacturing specifications, design parameters or other operating requirements without departing from the general principles of the same.

We claim:

1. A variable tint cosmetic composition, said composition comprising:
a substantially anhydrous carrier particle suitably adapted to carry a pigment material, wherein said carrier particle comprises a first color tint; and
a substantially anhydrous topical formulation, wherein said topical formulation is suitably adapted to at least one of suspend and substantially separate a plurality of anhydrous pigmentation carrier particles, wherein said topical formulation comprises a second color tint.

2. The composition of claim 1, wherein:
said first color tint comprises at least one of a first hue, a first saturation and a first brightness; and
said second color tint comprises at least one of a second hue, a second saturation and a second brightness.

3. The composition of claim 2, wherein at least one of:
said first hue is different from said second hue;
said first saturation is different from said second saturation; and
said first brightness is different from said second brightness.

4. The composition of claim 2, wherein at least one of:
said first hue is substantially opposite said second hue;
said first saturation is substantially equivalent to said second saturation; and
said first brightness is substantially equivalent to said second brightness.

5. The composition of claim 1, wherein said composition is suitably adapted to comprise at least one of a concealer, a foundation, a shadowing agent, a powder make-up and a liquid make-up.

6. The composition of claim 1, wherein said pigmentation carrier particle comprises at least 10% (wt/wt of carrier particle mass) of pigment material.

7. The composition of claim 1, wherein said anhydrous topical formulation comprises at least one of a jojoba ester, an ethylhexyl palmitate, petrolatum and polyethylene.

8. The composition of claim 1, wherein said pigmentation carrier particle is at least one of substantially soft, substantially spherical and substantially uniform.

9. The composition of claim 1, wherein said pigmentation carrier particle is suitably adapted to at least one of substantially disintegrate, rupture, burst and release said pigmentation material upon at least one of:
application of mechanical shear forces; and
agitation of said carrier particle.

10. The composition of claim 1, wherein said the average diameter of said pigmentation carrier particle is between about 100 to about 4000 microns.

11. The composition of claim 1, wherein said cosmetic composition is suitably adapted to dry more slowly than a conventional liquid base.

12. A method for at least one of providing, manufacturing or using a variable tint cosmetic product, said method comprising the steps of:
providing a substantially anhydrous carrier particle suitably adapted to carry a pigment material, wherein said carrier particle comprises a first color tint; and
providing a substantially anhydrous topical formulation, wherein said topical formulation is suitably adapted to at least one of suspend and substantially separate a plurality of anhydrous pigmentation carrier particles, wherein said topical formulation comprises a second color tint.

13. The method of claim 12, wherein:
said first color tint comprises at least one of a first hue, a first saturation and a first brightness; and
said second color tint comprises at least one of a second hue, a second saturation and a second brightness.

14. The method of claim 13, wherein at least one of:
said first hue is different from said second hue;
said first saturation is different from said second saturation; and
said first brightness is different from said second brightness.

15. The method of claim 13, wherein at least one of:
said first hue is substantially opposite said second hue;
said first saturation is substantially equivalent to said second saturation; and
said first brightness is substantially equivalent to said second brightness.

16. The method of claim 12, wherein the resulting composition is suitably adapted to comprise at least one of a concealer, a foundation, a shadowing agent, a powder make-up and a liquid make-up.

17. The method of claim 12, wherein said pigmentation carrier particle comprises at least 10% (wt/wt of carrier particle mass) of pigment material.

18. The method of claim 12, wherein said anhydrous topical formulation comprises at least one of a jojoba ester, an ethylhexyl palmitate, petrolatum and polyethylene.

19. The method of claim 12, wherein said pigmentation carrier particle is at least one of substantially soft, substantially spherical and substantially uniform.

20. The method of claim 12, wherein said pigmentation carrier particle is suitably adapted to at least one of substantially disintegrate, rupture, burst and release said pigmentation material upon at least one of:
application of mechanical shear forces; and
agitation of said carrier particle.

21. The method of claim 12, wherein said the average diameter of said pigmentation carrier particle is between about 100 to about 4000 microns.

22. The method of claim 12, wherein the resulting composition is suitably adapted to dry more slowly than a conventional liquid base.

23. A variable tint cosmetic system, comprising:
a substantially anhydrous carrier particle suitably adapted to carry a pigment material, wherein said carrier particle comprises a first color tint; and
a substantially anhydrous topical formulation, wherein said topical formulation comprises a second color tint.

24. The system of claim 23, wherein:
said first color tint comprises at least one of a first hue, a first saturation and a first brightness; and
said second color tint comprises at least one of a second hue, a second saturation and a second brightness.

25. The system of claim 24, wherein at least one of:
said first hue is different from said second hue;
said first saturation is different from said second saturation; and
said first brightness is different from said second brightness.

26. The system of claim 24, wherein at least one of:
said first hue is substantially opposite said second hue;
said first saturation is substantially equivalent to said second saturation; and
said first brightness is substantially equivalent to said second brightness.

27. The system of claim 23, wherein said system is suitably adapted to comprise at least one of a concealer, a foundation, a shadowing agent, a powder make-up and a liquid make-up.

28. The system of claim 23, wherein said pigmentation carrier particle comprises at least 10% (wt/wt of carrier particle mass) of pigment material.

29. The system of claim 23, wherein said anhydrous topical formulation comprises at least one of a jojoba ester, an ethylhexyl palmitate, petrolatum and polyethylene.

30. The system of claim 23, wherein said pigmentation carrier particle is at least one of substantially soft, substantially spherical and substantially uniform.

31. The system of claim 23, wherein said pigmentation carrier particle is suitably adapted to at least one of substantially disintegrate, rupture, burst and release said pigmentation material upon at least one of:
application of mechanical shear forces; and
agitation of said carrier particle.

32. The system of claim 23, wherein said the average diameter of said pigmentation carrier particle is between about 100 to about 4000 microns.
